(19) United States
(12) Patent Application Publication
(10) Pub. No.: US 2020/0261409 A1
(43) Pub. Date: Aug. 20, 2020

(54) METHODS FOR PAIN TREATMENT USING RESINIFERATOXIN

(71) Applicants: SORRENTO THERAPEUTICS, INC., San Diego, CA (US); Mayo Foundation for Medical Education and Research, Rochester, MN (US)

(72) Inventors: Mike Royal, Raliegh, NC (US); Bryan Jones, San Diego, CA (US); Andreas S. Beutler, Rochester, MN (US); Timothy P. Maus, Rochester, MN (US)

(21) Appl. No.: 15/776,239

(22) PCT Filed: Nov. 18, 2016
(86) PCT No.: PCT/US2016/062776

§ 371 Date: May 15, 2018

Related U.S. Application Data
(60) Provisional application No. 62/258,349, filed on Nov. 20, 2015.
(60) Provisional application No. 62/382,177, filed on Aug. 31, 2016.

(51) Int. Cl. A61K 31/357; A61K 9/00; A61K 31/445; A61P 29/00

(52) U.S. Cl. CPC A61K 31/357; A61P 29/00; A61K 31/445; A61K 9/0019

(57) ABSTRACT

Methods for treating pain using resiniferatoxin. The resiniferatoxin may be administered to certain anatomic sites using image-guided delivery. The resiniferatoxin may be administered in low doses in the range of 0.5 to 3.0 μg of resiniferatoxin in a human patient. The resiniferatoxin may cause the partial or total ablation of nerve afferents associated with the pain response.

### RELATED APPLICATIONS

This application claims priority to U.S. Provisional Patent Application Ser. Nos. 62/258,349, filed Nov. 20, 2015 and entitled “Methods for Lower Dose Pain Treatment Administration of Resinferatoxin” and 62/382,177, filed Aug. 31, 2016 and entitled “Selective Neurolysis and Analgesic Effects of Image-Guided Periganglionic Epidural Delivery of Resiniferatoxin,” the disclosures of which are hereby incorporated by reference in their entirety.

### TECHNICAL FIELD

The present disclosure relates to methods of delivering resiniferatoxin to patients with chronic pain, in particular the treatment of chronic pain patients by administration of resiniferatoxin by injection in or near the spine, such as epidural, periganglionic, intrathecal or intra-ganglionic injection.

### BACKGROUND

Direct application of a vanilloid receptor agonist into the neuron cell body contained in a ganglion opens calcium channels in VR1-expressing neuronal perikarya, triggering a cascade of events leading to cell death. Vanilloid receptor-1 (VR1) is a multimeric cation channel prominently expressed in nociceptive primary afferent neurons (Caterina et al., Nature 389:816-824, 1997; Tominaga et al., Neuron 531-543, 1998). Activation of the receptor typically occurs at the nerve endings via application of painful heat (VR1 transduces heat pain) or during inflammation or exposure to vanilloids. Activation of VR1 by an agonist, such as resiniferatoxin or capsaicin, results in the opening of calcium channels and the transduction of pain sensation (Szalllasi et al., Mol. Pharmacol. 56:581-587, 1999.) After an initial activation of VR1, VR1 agonists desensitize VR1 to subsequent stimuli. This desensitization phenomenon has been exploited in order to produce analgesia to subsequent nociceptive challenge. For example, it has been shown that topical administration of resiniferatoxin (RTX), which is a potent vanilloid receptor agonist, at the nerve endings in the skin triggers a long-lasting insensitivity to chemical pain stimulation. Furthermore, it has been shown that both subcutaneous and epidural administration of the RTX produce thermal analgesia when administered to rats, with no restoration of pain sensitivity for over 7 days (Szabo et al., Brain Res. 840:92-98, 1999).

Resiniferatoxin (RTX) is used as a vanilloid receptor agonist. RTX is unlike structurally related phorbol esters because it acts as an ultrapotent analog of capsaicin, the pungent principle of the red pepper. RTX is a tricyclic diterpene isolated from Eurphorbia resinifera. RTX induces pain, hypothermia, and neurogenic inflammation; the acute responses are followed by desensitization to RTX and by cross-desensitization to capsaicin. A homovanillyl group is an important structural feature of capsaicin and the most prominent feature distinguishing resiniferatoxin from typical phorbol-related compounds. Naturally occurring or native RTX has the following structure:

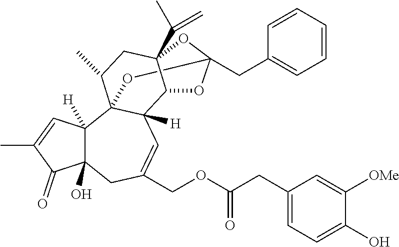

RTX and analog compounds such as tinyatoxin as well other compounds, (homovanillyl esters of diterpenes such as 12-deoxyphorbol 13-phenylacetate 20-homovanillate and mezerein 20-homovanillate) are described in U.S. Pat. Nos. 4,939,194; 5,021,450; and 5,232,684, the disclosures of which are incorporated by reference herein. Other resiniferatoxintype phorboid vanilloids have also been identified (Szallasi et al., Brit. J. Phrmacol. 128:428-434, 1999). As used herein, “a resiniferatoxin” or “an RTX” refers to naturally occurring RTX and analogs of RTX, including other phorbol vanilloids with VR1 agonist activity.

Prior publications indicate that the preferred dose for an adult human is about 25 μg of RTX for intrathecal administration in a total volume from about 0.5 mL to about 4.0 mL. Further, prior publications indicated that the preferred dose for intraganglionic administration for an adult human is about 5-20 μg in a volume of about 100 μL to about 200 μL. Prior studies have used multiple injections.

Although these previous methods may have certain value, there remains a need for a treatment capable of limiting the anatomic range of RTX and reducing side effects associated with the spread of RTX to non-targeted nerve locations. Moreover, given the difficulty of epidural, periganglionic, intrathecal, and intra-ganglionic injections, there is a need in the art to minimize the number of such injections as there is a significant risk of damage that could be done with each injection.

### SUMMARY

The present disclosure relates to methods of treating pain in animals and humans by administering resiniferatoxin to them by epidural delivery to target specific dorsal root ganglia (DRG) under fluoroscopic or CT guidance (periganglionic epidural). More particularly, for treatment of pain, injections may be made at lumbar or thoracic vertebral levels. The amount of resiniferatoxin administered in each administration may be varied and includes but is not limited to from about 500 ng to about 50 μg of resiniferatoxin. In some embodiments, the dose may be about 0.5 μg to about 3 μg. The resiniferatoxin may be diluted in a diluent and the amount of diluent may be varied and may be but is not limited to from about 100 μl to about 1000 μl of diluent. The diluent may also include excipients and/or other analgesics, such as local anesthetics or opioids. The resiniferatoxin may be delivered by infusion where the infusion flow rate may be varied and includes but is not limited to a flow rate of from about 10 μL/min to about 500 μl/min. The administration of the resiniferatoxin may include more than one target DRG level by periganglionic epidural delivery. Any targeted DRG need only receive a single administration of resiniferatoxin.

The present disclosure relates to the administration of resiniferatoxin by the periganglionic epidural route to induce apoptosis (programmed cell death) of TRPV1 expressing DRG neurons. This process results in a reduction in substance P (sP) production, as shown by reduced histological staining for sP. The present disclosure also relates to methods of treating pain in animals and humans by conducting imaging of the spinal target level of the animal or human; percutaenously inserting a spinal needle into the lateral periganglionic epidural space; verifying the position of the needle by imaging and/or injection of contrast media; and delivering resiniferatoxin to the periganglionic epidural space.

The methods of the present disclosure provide more localized ablation of dorsal root ganglion and provide a lower dose profile than prior art methods. The present disclosure provides a method for single dose administration of resiniferatoxin (RTX) for pain indications. More specifically the disclosed method is for epidural-periganglionic administration of a lower dose amount of RTX in the range of 0.2 to 3.0 μg of RTX per human adult, and a single dose administration. The injection site is in or near the spine, and selected from the group consisting of epidural, periganglionic, intrathecal, and intra-ganglionic.

### BRIEF DESCRIPTION OF DRAWINGS

FIGS. 1A and 1B depict the lumbar spine of a pig with arrows indicating features relevant to image-guided transforaminal (TF) injection. FIGS. 1A and 1B provide anatomical specificity for selective neurolysis (SN) by image-guided cannulation of the periganglionic epidural (PG-ED) space by showing the lumbosacral spine anatomy in a 3-dimensional (3D) reconstruction. FIG. 1A shows a sagittal view of lumbar neural foramina in a pig (arrows with stems) accessible by the TF approach at all levels down to L4. At the L5 level the posterolateral aspect of the neural foramen was covered by the iliac crest (arrowhead) in approximately 40% of animals. Neural foramina at the sacral level were located ventrally (not shown) rendering them inaccessible to TF injection. FIG. 1B shows a coronal view of the wide interlaminar opening (arrows with stems) at the lumbar vertebral levels. The sacrum shows prominent central dorsal foramina (full arrowheads) and rudimentary lateral dorsal foramina (outlined arrows with stems);

FIGS. 2A-2F show injection path and contrast using TF (FIGS. 2A-2C) and interlaminal (IL) (FIGS. 2D-2F) routes. A TF cannulation is shown (L4) in FIGS. 2A-2C. The delivery device was placed into the PG-ED space (FIG. 2A). 0.2 mL of diluted contrast media were injected achieving PG-ED spread (FIG. 2B). Injection of 0.5 mL of RTX solution (or control vehicle) diluted the contrast in the PG-ED compartment, thereby verifying that the injectate had been delivered ED (FIG. 2C). An IL cannulation is shown (51) in FIGS. 2D-2F depicting needle placement (FIG. 2D), contrast spread displaying some flow to the ventral intraspinal space (FIG. 2E), and contrast dilution by the injectate (FIG. 2F).

FIGS. 3A-3D show complete, selective knock-out of primary afferent pain fibers by “selective” neurolysis (SN). Immunohistochemistry staining for sP (green) and neuronal nuclear antigen (NeuN-red) showed that SN resulted in complete specific destruction of peptidergic afferent pain fibers. FIGS. 3A-3B: sP+ positive fibers (green) are seen terminating in the superficial laminae of the dorsal horns of the spinal cord (SC) in a control animal (FIG. 3A). FIG. 3B shows histological staining of the dorsal horn of a subject treated with RTX to achieve SN which demonstrates no sP staining. The centripetal axonal terminals of the nociceptive primary sensory neurons, whose cell bodies are located in the DRG, were lost. The effect was complete in all animals receiving RTX. SC neurons were unaffected as indicated by similar results of staining with NeuN (red). FIGS. 3C-3D show that motor neurons in the anterior horn of the SC were unaffected. Scale bars=200 μm.

FIGS. 4A-4F show histological staining for sP in the dorsal horn of control animals (FIGS. 4B, 4C, and 4F) and of animals treated with RTX according to the present disclosure (FIGS. 4A, 4D, and 4E). All of the RTX-treated animals show complete loss of sP+ fibers (green) in the dorsal horns and all control animals showed that sP+ axons were intact.

### DETAILED DESCRIPTION

### Definitions

The terms “subarachnoid space” and cerebral spinal fluid (CSF) space incorporate the common usage refers to the anatomic space between the pia mater and the arachnoid membrane containing CSF.

The term “intrathecal administration” means the administration of a composition directly into the spinal subarachnoid space.

“Intraganglionic administration” means administration to a ganglion. Intraganglionic administration can be achieved by direct injection into the ganglion and also includes selective nerve root injections, in which the compound passes up the connective tissue sleeve around the nerve and enters the ganglion from the nerve root just outside the vertebral column.

“Epidural” means on or around the dura mater particularly of the spinal cord. For example, epidural delivery may include delivery to the epidural space without direct injection into nerves or may include epidural delivery into nerve tissue.

The current technology provides for effective pain relive through neurolysis, nerve ablation, or both by RTX injection at specific epidural sites as guided by imaging technology. Intraganglionic administration may be used in conjunction with an imaging technique such as MRI or x-ray contrast dyes or agents, to visualize the targeted ganglion and area of administration. Clinical practice will generally desire as few injections as possible, and an opportunity to optimize drug delivery while achieving a rigorous outcome may be particularly beneficial in the case of a treatment involving neurolysis or nerve ablation. The present technology achieves this result the studies in pigs reported herein show that RTX injection according to the current technology achieved significant pain reduction using reduced amounts of RTX at specifically targeted injection sites, reducing the risk of unnecessary or undesirable neurolysis or nerve ablation. The experiments disclosed herein also show that delivering a lower amount of RTX, making a single injection to deliver the RTX, or combining the two to make a single injection with a lower amount of RTX, may provide effective treatment. By contrast, prior studies used multiple injections with higher cumulative doses of RTX in order to show a reduction in pain. The present disclosure relates to methods of treating pain in animals and humans by administering resiniferatoxin to them by periganglionic epidural delivery.

Resiniferatoxin (RTX) is a common name for (1R, 6R, 13R, 15R, 17R)-13-benzyl-6hydroxy-4,17-dimethyl-oxo-15-(prop-1-en-2-yl)-12,14,18-trioxapentacyclo-octadeca-3,8-dien-8-yl]methyl 2-(4-hydroxy-3-methoxyphenyl)acetate. RTX may be obtained as a naturally occurring product from the resin spurge, and has been synthetically prepared. (Wender, P. A.; et al. “The First Synthesis of a Daphnane Diterpene: The Enantiocontrolled Total Synthesis of (+)-Resiniferatoxin” 119 J. Am. Chem. Soc. 52, 12976-12977 (1997)). Resiniferatoxin is a potent analog of capsaicin, which is found in chili peppers. Resiniferatoxin depletes the sensory neuropeptide substance P (sP) due to a combination of neuron loss and decreased sP synthesis in the surviving neurons. Without being bound by theory, the postulated mechanism-of-action of RTX is inhibition of transient receptor potential vanilloid type 1 receptor (TRPV1) on targeted tissue.

The present disclosure relates to a “selective” neurolysis (SN) procedure in large animals using resiniferatoxin administered via the periganglionic epidural (PG-ED) route. SN, which may be known as “Dual-Selective Nociceptive Neurolysis,” may be achieved by administering resiniferatoxin using image guided nerve root-selective delivery. Imagine guided nerve root-selective delivery is a procedure requiring a trained subspecialized physician using image guidance. Because of the cost and complexity of SN, it may be impractical or uneconomical to use SN methods with traditional pain therapies like local anesthetics or corticosteroids, as these traditional therapies generally have at best a short-lived effect and are therefore poor choices in terms of the overall value of health outcomes for patients. “Value” is an increasingly important benchmark for payers such as Medicare and health insurers. SN with RTX, on the other hand, may provide a long-lasting or permanent treatment by inducing irreversible nociceptive neurolysis or nerve ablation. It is therefore a high-value, “one-and-done” intervention for treating severe chronic pain. To realize this potential value, the SN treatment must be tailored to limit the risk of RTX spreading to distant sites via the cerebrospinal fluid and to limit neurolysis to targeted segments.

Administration of RTX may be performed in conjunction with an image guidance procedure. For example, administration may be performed using image analysis with CAT scan, fluoroscopy, open MRI, and x-ray (using x-ray contrast dyes). RTX can also be administered intrathecally, typically in an isobaric or hyperbaric pharmaceutically acceptable excipient as further described below. Typically, RTX administered to a particular ganglion (T1-T4) is administered to create a temporary environment from about 1 to 5 minutes. For intraganglionic administration to a dorsal root or autonomic ganglion, a typical volume injected is from about 50 to 300 microliters delivering a total amount of RTX that rages from about 50 ng to about 50 micrograms. Often the amount administered is from 200 ng to 1 μg. RTX can be administered as a bolus or infused over a period of time, typically from 1 to 5 minutes. For intraganglionic administration to a trigeminal ganglion, a volume of from about 100 microliters to about 500 microliters is typically used to deliver from about 50 ng to about 50 μg of RTX. RTX can be infused over a length of time from about 1 to 5 minutes, or can be delivered as one or more boluses. Dosages in the ranges of 100 ng to 500 1 μg are often used. For intrathecal administration, an amount from about 0.5 to 5 ml, often 3 ml, is injection into the subarachnoid space. The total amount of RTX in the injected volume is usually from about 500 ng to about 500 μg.

Prior to the methods of the present disclosure it was unknown whether PG-ED administered resiniferatoxin would be able to reach targeted neural structures such as DRG neurons and their centripetal processes in large animals and, by extension, humans. While epidural administration of resiniferatoxin (and capsaicin) have been performed in rats (Elmerl D, Papiri-Kricheli D. Epidural capsaicin produces prolonged segmental analgesia in the rat. Exp Neurol 1987; 97:169-78; Szabo T, Olah Z, Iadarola M J, Blumberg P M. Epidural resiniferatoxin induced prolonged regional analgesia to pain. Brain Res 1999; 840:92-8), the epidural administration was performed using a surgical incision to place a catheter within the lumbar epidural space without the use of imaging guidance. Results of these studies have been inconsistent possibly due to differences in permeability in rat dural membranes that allows for more facile penetration into the intrathecal space. Additionally, the epidural space is less well defined in rodents compared to larger animals and humans and no attempt was made to target neural structures unilaterally.

Availability of large animal models for image guided PG-ED injections is limited, in part because PG-ED delivery of corticosteroids was developed in the 1960s without preclinical studies (D. Nelson, W. M. Landau, Intraspinal steroids: history, efficacy, accidentality, and controversy with review of United States Food and Drug Administration reports, J. Neurol. Neurosurg. Psychiatry 70, 433-443 (2001)). Some work on an epidural route of RTX administration has been attempted in rats but no attempts to utilize an image-guided PG-ED route in large animals has previously been conducted. The methods of the present disclosure have been developed for PG-ED injection under computed tomography fluoroscopy (CTF) for large animals including but not limited to swine, horses, cows, sheep and humans, but could also be performed using x-ray fluoroscopic guidance. The methods of the present disclosure utilize resiniferatoxin PG-ED administration to lead to segmental knock-out of sP+ pain fibers in the spinal cord proving elimination of the centripetal projection of TRPV1 neurons, i.e., SN. Finally we evaluated, whether SN could improve pain-related functional outcomes by clinical assessment in a swine model of knee osteoarthritis.

The methods of the present disclosure provide for administering various amounts of resiniferatoxin. In embodiments, the present technology allows reduced quantities of RTX, on the order of about 0.5 μg to about 3.0 μg, to achieve effective pain reduction. The amount of resiniferatoxin may be administered in a single administration or in more than one administration can be from about 10 ng to about 100 μg or from about 10 ng to about 90 μg or from about 10 ng to about 80 μg or from about 10 ng to about 70 μg or from about 10 ng to about 60 μg or from about 10 ng to about 50 μg or from about 10 ng to about 40 μg or from about 10 ng to about 30 μg or from about 10 ng to about 20 μg or from about 10 ng to about 10 μg or from about 10 ng to about 5 μg or from about 10 ng to about 1 μg or from about 50 ng to about 100 μg or from about 50 ng to about 90 μg or from about 50 ng to about 80 μg or from about 50 ng to about 70 μg or from about 50 ng to about 60 μg or from about 50 ng to about 50 μg or from about 50 ng to about 40 μg or from about 50 ng to about 30 μg or from about 50 ng to about 20 μg or from about 50 ng to about 10 μg or from about 50 ng to about 5 μg or from about 10 ng to about 1 μg or about 100 ng to about 100 μg or from about 100 ng to about 90 μg or from about 100 ng to about 80 μg or from about 100 ng to about 70 μg or from about 10 ng to about 60 μg or from about 100 ng to about 50 μg or from about 100 ng to about 40 μg or from about 100 ng to about 30 μg or from about 100 ng to about 20 μg or from about 100 ng to about 10 μg or from about 100 ng to about 5 μg or from about 100 ng to about 1 μg or from about 200 ng to about 100 μg or from about 200 ng to about 90 μg or from about 200 ng to about 80 μg or from about 200 ng to about 70 μg or from about 200 ng to about 60 μg or from about 10 ng to about 200 μg or from about 200 ng to about 40 μg or from about 200 ng to about 30 μg or from about 200 ng to about 20 μg or from about 200 ng to about 10 μg or from about 200 ng to about 5 μg or from about 200 ng to about 1 μg or from about 500 ng to about 100 μg or from about 500 ng to about 90 μg or from about 500 ng to about 80 μg or from about 500 ng to about 70 μg or from about 500 ng to about 60 μg or from about 500 ng to about 50 μg or from about 500 ng to about 40 μg or from about 500 ng to about 500 μg or from about 500 ng to about 20 μg or from about 500 ng to about 10 μg or from about 500 ng to about 5 μg or from about 500 ng to about 1 μg.

The methods of the present disclosure provide for administering resiniferatoxin at various rates. The rate of administration of resiniferatoxin can be from about 1 μl/min to about 1000 μl/min or from about 1 μl/min to about 900 μl/min or from about 1 μl/min to about 800 μl/min or from about 1 μl/min to about 700 μl/min or from about 1 μl/min to about 600 μl/min or from about 1 μl/min to about 500 μl/min or from about 1 μl/min to about 400 μl/min or from about 1 μl/min to about 300 μl/min or from about 1 μl/min to about 200 μl/min or from about 1 μl/min to about 100 μl/min or from about 5 μl/min to about 1000 μl/min or from about 5 μl/min to about 900 μl/min or from about 5 μl/min to about 800 μl/min or from about 5 μl/min to about 700 μl/min or from about 5 μl/min to about 600 μl/min or from about 5 μl/min to about 500 μl/min or from about 5 μl/min to about 400 μl/min or from about 5 μl/min to about 300 μl/min or from about 5 μl/min to about 200 μl/min or from about 5 μl/min to about 100 μl/min or from about 10 μl/min to about 1000 μl/min or from about 10 μl/min to about 900 μl/min or from about 10 μl/min to about 800 μl/min or from about 10 μl/min to about 700 μl/min or from about 10 μl/min to about 600 μl/min or from about 10 μl/min to about 500 μl/min or from about 10 μl/min to about 400 μl/min or from about 10 μl/min to about 300 μl/min or from about 10 μl/min to about 200 μl/min or from about 10 μl/min to about 100 μl/min or from about 50 μl/min to about 1000 μl/min or from about 50 μl/min to about 900 μl/min or from about 50 μl/min to about 800 μl/min or from about 50 μl/min to about 700 μl/min or from about 50 μl/min to about 600 μl/min or from about 50 μl/min to about 500 μl/min or from about 50 μl/min to about 400 μl/min or from about 50 μl/min to about 300 μl/min or from about 50 μl/min to about 200 μl/min or from about 50 μl/min to about 100 μl/min or from about 100 μl/min to about 1000 μl/min or from about 100 μl/min to about 900 μl/min or from about 100 μl/min to about 800 μl/min or from about 100 μl/min to about 700 μl/min or from about 100 μl/min to about 600 μl/min or from about 100 μl/min to about 500 μl/min or from about 100 μl/min to about 400 μl/min or from about 100 μl/min to about 300 μl/min or from about 100 μl/min to about 200 μl/min.

The methods of the present disclosure provide for administering resiniferatoxin at various volumes. The volume of the solution containing for a single administration of resiniferatoxin can be from about 100 μl to about 2000 μl or from about 100 μl to about 1900 μl or from about 100 μl to about 1800 μl or from about 100 μl to about 1700 μl or from about 100 μl to about 1600 μl or from about 100 μl to about 1500 μl or from about 100 μl to about 1400 μl or from about 100 μl to about 1300 μl or from about 100 μl to about 1200 μl or from about 100 μl to about 1100 μl or from about 100 μl to about 1000 μl or from about 100 μl to about 900 μl or from about 100 μl to about 800 μl or from about 100 μl to about 700 μl or from about 100 μl to about 600 μl or from about 100 μl to about 500 μl or from about 100 μl to about 400 μl or from about 100 μl to about 300 μl or from about 100 μl to about 200 μl or from 200 μl to about 2000 μl or from about 200 μl to about 1900 μl or from about 200 μl to about 1800 μl or from about 200 μl to about 1700 μl or from about 200 μl to about 1600 μl or from about 200 μl to about 1500 μl or from about 200 μl to about 1400 μl or from about 200 μl to about 1300 μl or from about 200 μl to about 1200 μl or from about 200 μl to about 1100 μl or from about 200 μl to about 1000 μl or from about 200 μl to about 900 μl or from about 200 μl to about 800 μl or from about 200 μl to about 700 μl or from about 200 μl to about 600 μl or from about 200 μl to about 500 μl or from about 200 μl to about 400 μl or from about 200 μl to about 300 μl or from about 300 μl to about 2000 μl or from about 300 μl to about 1900 μl or from about 300 μl to about 1800 μl or from about 300 μl to about 1700 μl or from about 300 μl to about 1600 μl or from about 300 μl to about 1500 μl or from about 300 μl to about 1400 μl or from about 300 μl to about 1300 μl or from about 300 μl to about 1200 μl or from about 300 μl to about 1100 μl or from about 300 μl to about 1000 μl or from about 300 μl to about 900 μl or from about 300 μl to about 800 μl or from about 300 μl to about 700 μl or from about 300 μl to about 600 μl or from about 300 μl to about 500 μl or from about 300 μl to about 400 μl or from about 400 μl to about 2000 μl or from about 400 μl to about 1900 μl or from about 400 μl to about 1800 μl or from about 400 μl to about 1700 μl or from about 400 μl to about 1600 μl or from about 400 μl to about 1500 μl or from about 400 μl to about 1400 μl or from about 400 μl to about 1300 μl or from about 400 μl to about 1200 μl or from about 400 μl to about 1100 μl or from about 400 μl to about 1000 μl or from about 400 μl to about 900 μl or from about 400 μl to about 800 μl or from about 400 μl to about 700 μl or from about 400 μl to about 600 μl or from about 400 μl to about 500 μl or from about 500 μl to about 2000 μl or from about 500 μl to about 1900 μl or from about 500 μl to about 1800 μl or from about 500 μl to about 1700 μl or from about 500 μl to about 1600 μl or from about 500 μl to about 1500 μl or from about 500 μl to about 1400 μl or from about 500 μl to about 1300 μl or from about 500 μl to about 1200 μl or from about 500 μl to about 1100 μl or from about 500 μl to about 1000 μl or from about 500 μl to about 900 μl or from about 500 μl to about 800 μl or from about 500 μl to about 700 μl or from about 500 μl to about 600 μl.

Resiniferatoxin can be administered alone or as admixtures with conventional excipients, for example, pharmaceutically or physiologically acceptable organic or inorganic carrier substances which do not deleteriously react with the composition. Suitable pharmaceutically acceptable carriers include water, salt solutions (such as Ringer's solution), alcohols, oils, gelatins and carbohydrates (such as lactose, amylose or starch), fatty acid esters. Such preparations can be sterilized and, if desired, mixed with auxiliary agents such as lubricants, preservatives, stabilizers, wetting agents, emulsifiers, salts for influencing osmotic pressure, buffers, coloring and/or aromatic substances and the like which do not deleteriously react with the compositions administered to the patient. The resiniferatoxin may be solubilized in a diluent which may also include excipients and/or other analgesics, such as local anesthetics or opioids. In some embodiments resiniferatoxin may be administered in conjunction with a local anesthetic such as, but not limited to, dibucaine, bupivacaine, ropivacaine, procaine, lidocaine, xylocaine and others. The compositions of resiniferatoxin may be sterilized or lyophilized and reconstituted prior to use.

While resiniferatoxin may be administered at several target locations as described herein, epidural administration may offer particular advantages. For example, a medical professional may find an epidural injection to be faster, easier, safer, and more cost-effective than certain other forms of administration. Further, administration to the epidural space may be associated with a reduction in the migration of RTX within the spinal column. For example, RTX injected into the spinal fluid may tend to exhibit greater migration due to the characteristics of the fluid, whereas the tissue of the epidural space may exhibit less migration. Where migration of RTX occurs, the RTX may cause ablation of nerve afferents in other parts of the spinal column not targeted by the therapy.

In certain embodiments, the present technology may be practiced using a relatively small amount of RTX, such as about 0.2 μg to about 1.0 μg or about 0.5 μg to 3.0 μg. Such amounts of active are lower than has previously been taught. The present disclosure shows that these low amounts do show a significant reduction in pain and may represent sufficient pain relief to be an effective therapy for chronic pain patients. Achieving these results with a smaller quantity of RTX presents several advantages. In particular, because of the potential for migration of RTX, especially if injection is to be made into the spinal fluids, as discussed above, the RTX may migrate to other locations within the spine and may tend to ablate nerves within those areas of the spine. Using a smaller amount of RTX may reduce the extent of migration and the extent of ablation of non-targeted nerves. Additionally, the use of a small amount of RTX may support the use of a smaller volume of solution (assuming similar concentrations). Reducing the total amount of solvent injected may reduce side effects associated with the treatment.

### Example 1: Methods

Animals.

The study was approved by the Institutional Animal Care and Use Committee of the Mayo Clinic. Female Yucatan miniature swine (Exemplar Genetics, Sioux Center, Iowa, USA) weighing 25-45 kg at the time of RTX administration were used. For both procedures (i.e., ACLT and PG-ED injection), the pigs were anesthetized by intramuscular injection of Telazol (tiletamine and zolazepam, 5 mg/kg; Fort Dodge Animal Health, Fort Dodge, Iowa, USA), xylazine (2 mg/kg; Akorn, Decatur, Ill., USA), and glycopyrrolate (0.01 mg/kg; Baxter Healthcare, Deerfield, Ill., USA), followed by endotracheal intubation and anesthesia maintenance with 1.5%-2% isoflurane (Piramal Healthcare, Bethlehem, Pa., USA). For antibiotic prophylaxis, the animals were given a single dose of intramuscular ceftiofur (5 mg/kg; Pfizer, New York, N.Y., USA). For postoperative analgesia, the animals received a single dose of slow release buprenorphine (0.18 mg/kg; Wildlife Pharmaceuticals, Windsor, Colo., USA) intramuscularly and then oral carprofen (3 mg/kg; Pfizer Animal Health, New York, N.Y., USA) as needed for the first three postoperative days. For CTF guided PG-ED injection of RTX, a clinical CT scanner (Definition DS, Siemens Healthcare) with interventional CTF hardware and software packages was used. A full CT scan of the lumbosacral 13 spine was first performed to visualize the spinal anatomy and to identify the optimal access routes to the targeted PG-ED segments. The CT scanner acquisition settings were the same as reported previously (J. Pleticha, et al., Pig lumbar spine anatomy and imaging-guided lateral lumbar puncture: A new large animal model for intrathecal drug delivery, J. Neurosci. Methods 216, 10-15 (2013)). Next, a 22 G, 6″ spinal needle with Quincke tip (Kimberly-Clark, Roswell, Ga., USA) was advanced to the PG-ED space following the predetermined trajectory. The PG-ED position of the tip was verified by a slow injection of 0.25 mL of iodinated contrast media (Omnipaque 240, Novation, Chicago, Ill., USA), diluted in normal saline and bupivacaine (Hospira, Lake Forrest, Ill., USA) in a ratio of 2:5:3. The RTX was delivered by slow infusion using an injection apparatus described previously for intraganglionic delivery (J. Pleticha, et al., Minimally invasive convection-enhanced delivery of biologics into dorsal root ganglia: validation in the pig model and prospective modeling in humans, J. Neurosurg. 121, 851-858 (2014)). At each spinal level, 5 μg RTX diluted in 500 μl diluent containing 0.25% bupivacaine was infused at a flow rate of 100 μl/min. Control animals received matching volume of the vehicle only. The needle was left in place for an additional three minutes and withdrawn. The true PG-ED delivery of the injectate was confirmed by repeat CTF imaging showing dilution of the contrast media within the PG-ED space. The animals were euthanized by intravenous injection of pentobarbital (Vortech Pharmaceuticals, Dearborn, Mich., USA). The spinal cord was harvested, fixed in 4% paraformaldehyde for 24 hours, and stored in phosphate-buffered saline (PBS) at 4° C. until further analysis.

Immunohistochemistry. The spinal cord samples were embedded in paraffin, sectioned at 10 μm 14 thickness, and adhered to slides (Fisher SuperFrost Plus, Waltham, Mass., USA). Tissue slides were deparaffinized, rehydrated with water and rinsed in PBS. Slides were incubated for 10 minutes at 90° C. in Target Retrieval Solution ((Dako, Carpinteria, Calif., USA) followed by repeated rinses in PBS. Nonspecific binding was blocked with 5% normal goat serum (Vector Laboratories, Burlingame, Calif., USA) diluted in PBS and sections were incubated for 1 hour with Streptavidin/Biotin Blocking Kit (Vector Laboratories, Burlingame, Calif., USA). The rabbit anti-sP (1:500) and chicken anti-NeuN (1:500) primary antibodies (Millipore, Billerica, Mass., USA) were prepared in BSA-PBS and applied to slides for overnight incubation. After 3 rinses for 10 minutes each in PBS, biotinylated anti-chicken IgG secondary antibodies (Vector Laboratories, Burlingame, Calif., USA) (1:500) were applied to the tissue and allowed to incubate for 1 hour. Slides were again rinsed 3 times in PBS for 10 minutes each. Slides were next incubated for 1 hour with Alexa Fluor 555 Streptavidin (1:1000) (Molecular Probes, Eugene, Oreg.) to label the NeuN and Alexa Fluor 488 goat anti-rabbit IgG antibodies (1:1000) (Molecular Probes, Eugene, Oreg.) to label sP. Following 3 final PBS rinses, glass coverslips were applied using ProLong Gold Antifade Reagent (Molecular Probes, Eugene, Oreg.). Tissue incubations and rinses were carried out at room temperature in Sequenza slide staining racks (Thermo Scientific, Waltham, Mass., USA). All primary and secondary antibody and fluorescent dye conjugate dilutions were prepared in 1% Bovine Serum Albumin in PBS. Primary antibody specificity was confirmed by including an isotype control. Nonimmune rabbit IgG antibodies (Vector Laboratories, Burlingame, Calif., USA) were diluted to the same concentration as the sP antibody and some tissue samples were 15 incubated in this solution in lieu of the primary antibody application.

Fluorescent Microscopy.

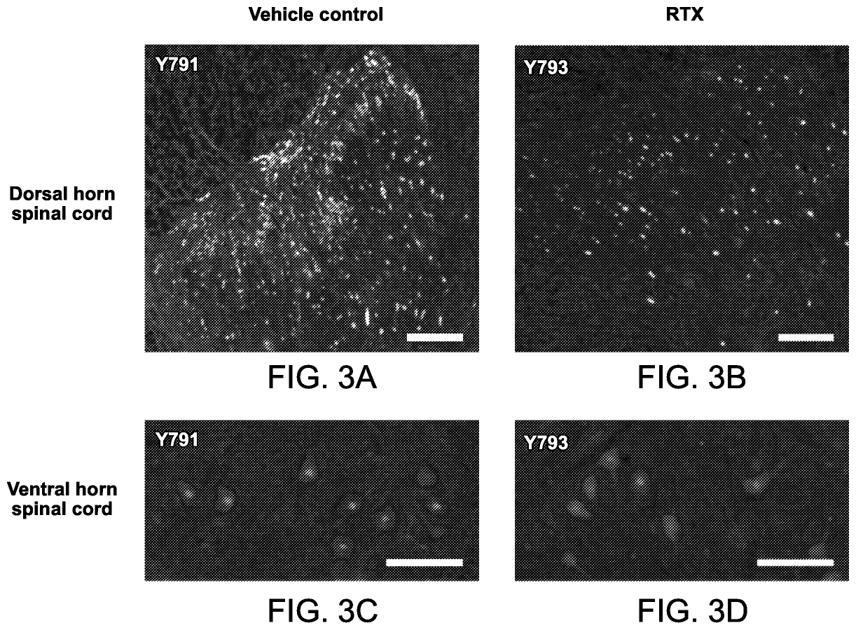

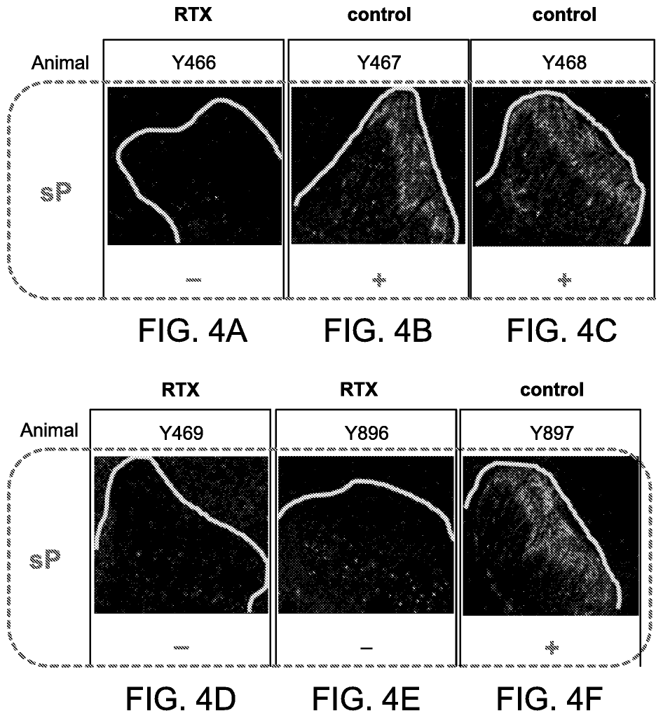

For FIGS. 3A-3D, images were taken using the LSM 780 confocal system (Carl Zeiss Microscopy, Jena, Germany). Images were acquired with a 20× objective in the lambda stack mode. The emission spectra of the respective fluorophores (Alexa 488 for sP and Alexa 555 for NeuN) were distinguished from tissue auto-fluorescence by linear unmixing. For FIGS. 4A-4F, images were taken for regions of interest using the Leica TCS SP5 Confocal System (Leica Microsystems, Inc., Buffalo Grove, Ill.). Sequential scanning with a 20× HCX PL APO CS objective of the sP fluorophore was performed using the same settings. For both instruments, the tile scan function was used to stitch together larger regions of interest.

Anterior Cruciate Ligament Transection (ACLT).

A vertical midline incision through the skin, subcutaneous fascia, and the patellar ligament was performed immediately below the distal edge of the patella to enter the capsule of the knee joint. The anterior cruciate ligament was then exposed by blunt dissection and a 2 mm portion of the ligament was excised. The completeness of the ACLT was confirmed by positive Lachmann test. The incision was closed in three layers and covered by DermaBond Prineo skin closure system (Ethicon, Cincinnati, Ohio, USA). In each pig, the side of ACLT procedure was randomly selected and skin incision only was performed on the contralateral side to allow blinded assessment of the resulting limp. Starting the second postoperative week, all animals underwent biweekly exercise schedule, consisting of 1-hour session of trotting in the hallway for reward, in order to facilitate the use of the affected knee and thereby cause progression of knee degeneration.

Behavioral Assessment of the Limp.

The animals were conditioned to walk at a steady pace following experimenters' guide. Three 30 s video clips were recorded 2-4 weeks prior to RTX administration (corresponding to 12-14 weeks after ACLT when the limp was manifest in all animals) and then 3-5 weeks after RTX administration. The three video clips before and after RTX administration were then paired and evaluated by three blinded observers, who independently decided whether the limp was unchanged in both sets or improved in one set compared to the other. An independent person from a different laboratory blinded the observers to the treatment.

SN on Knee Osteoarthritis (OA)-Related Behavior and Correlation with sP-Loss.

To assess the functional outcome of SN in a clinically relevant animal model, the procedure was used to treat swine with gait asymmetry due to chronic knee pain induced by unilateral anterior cruciate ligament transection (ACLT). In a randomized block design, n=3 pigs received SN with RTX and n=3 received a vehicle control. Behavioral assessment by blinded observers correctly identified all animals receiving the RTX by observing improvements in symmetric weight bearing (ri), while the gait of control animals was unchanged.

Statistical Analysis.

All study outcomes were binary such as “sP+” vs. “sP-” and “improved” vs. “unchanged”. A sign test was performed with the null hypothesis that both outcomes are equally likely.

### Example 2: Clinical Periganglionic Epidural (PG-ED) Drug Delivery Modeled in a Large Animal Species

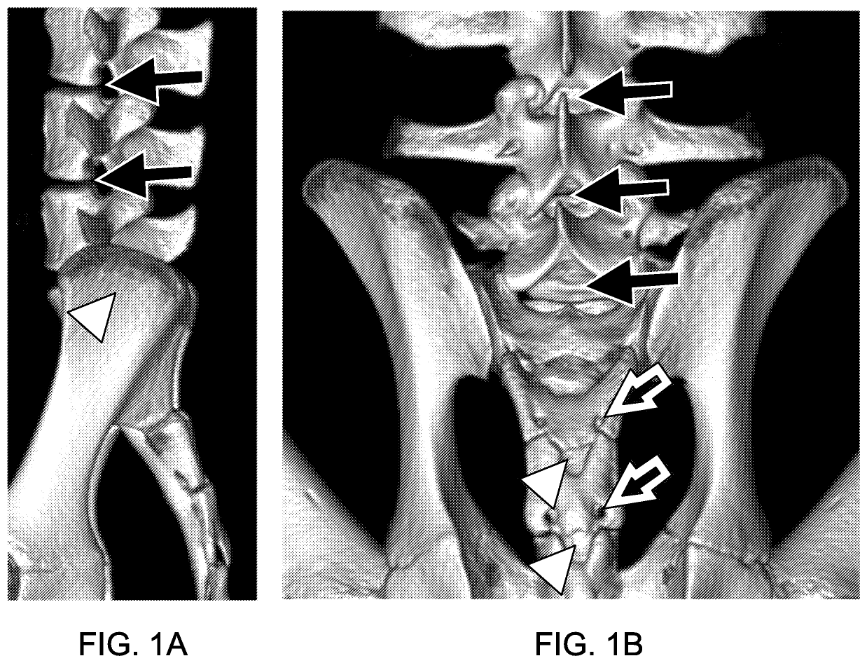

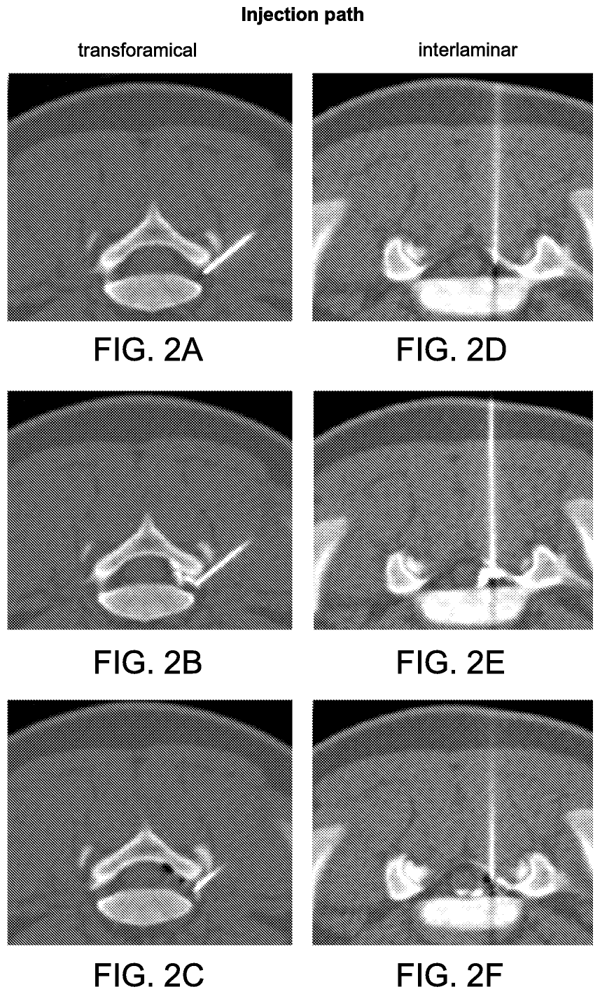

PG-ED drug delivery via swine neural foramina and interlaminar spaces corresponded closely to the human, differing primarily in the bulkier skeletal structure and the lack of paired dorsal sacral foramina (See generally FIGS. 1A-1B for relevant porcine skeletal structure). Multi-slice pulsed CT fluoroscopic guidance, as in human PG-ED injections, documented ED drug delivery and excluded intravascular, subdural or intrathecal distribution. A transforaminal (TF) approach, equivalent to TF human epidural injections, was utilized at all L4 segments and in 60% of L5 segments (See FIGS. 2A-2F). The TF route resulted in centripetal contrast flow to the intraspinal ED space; contrast crossed the midline in 50% of injections, but no rostrocaudal flow to adjacent segments was observed. A central route, equivalent to a human interlaminar injection (IL) was used in 40% of L5 segments and at all sacral levels. The central route yielded contrast flow to the subadjacent spinal level in 70% and contralateral spread via both the ventral and dorsal ED space also in 70% of animals.

### Example 3: Knock-Out of Substance P (sP)-Neurons from Posterior Roots

PG-ED injection of RTX resulted in a complete loss of sP+ axons in the dorsal horn of the spinal cord at the injected levels (compare FIG. 3A to FIG. 3B). The sP+ structures in the spinal cord anatomically correspond to the centripetal axonal terminals of the primary sensory neurons whose somata reside in the targeted DRG. A blinded analysis of the spinal cord samples revealed an ‘all or nothing’ effect, whereby the population of sP+ neurons was eliminated in all animals receiving RTX at all injected spinal levels but remained intact in all animals receiving placebo. (See FIGS. 3A-3B, 4A-4F). A concurrent neuronal nuclei (NeuN) staining was not affected by injection of either RTX or placebo, indicating that only the terminals of the nociceptive primary afferent were knocked out by RTX and that other neuronal populations in the spinal cord (e.g., the primary motor neurons) remained intact. (See FIGS. 3C-3D). The elimination of the sP+ neurons by PG-ED RTX was therefore complete, histologically selective, covered all injected spinal levels, and was found in all animals receiving the active drug, thereby establishing this procedure as SN.

### Example 4: Blinded Randomized Assessment of Gait Asymmetry Reversal after Pg-Ed Rtx

Unilateral anterior cruciate ligament transection (ACLT) resulted in development of knee osteoarthritis (OA) in the operated extremity. The OA manifested as profound limp, indicating weight-sparing of the affected porcine extremity. While the limp appeared immediately after the procedure (corresponding to acute postoperative pain), it remained sustained for at least 18 weeks with regular exercise, suggesting that a transition into a chronic pain state had occurred. To validate that the limp was pain related, animals were administered intramuscular morphine, which led to diminished weight-bearing asymmetry (as assessed by unblinded observations).

In animals with manifest gait asymmetry secondary to ACLT, SN was performed at the ipsilateral L4, L5, 51, and S2 spinal levels. In a randomized block 8 design, n=3 animals were given PG-ED RTX and n=3 animals were given inactive diluent (placebo control). Drug vials were labeled with a code by a research professional from a laboratory who was not involved in the study. The authors of the study were thereby unaware of the RTX vs. control assignment of individual animals. Blinded comparison of pig gait before and after the injection showed improvement of limp in all animals receiving RTX and no change or worsening of the limp in all animals receiving the inactive control, corresponding to the histological staining results shown in FIGS. 4A-4E.

### Example 5: Histological Verification of SN in ACLT Animals

The subsequent histological analysis of the dorsal horn of the spinal cord at injected spinal levels correlated perfectly with RTX vs. control status and with the behavioral data. As in the initial group of histologically assessed animals (above), ACLT animals showed complete loss of the sP+ axonal terminals in all animals that had received RTX and thereby had shown limb improvement, whereas the sP immunoreactivity was intact in all animals that had received the placebo and had remained unchanged in terms of pain behavior (FIGS. 4A-4E).

### Example 6: Statistical Strength Study Results in Example 1 to 5

The primary endpoint of the study was decrease of sP-immunoreactivity in the posterior horn of the spinal cord at the level of injected segments. This endpoint was tested in n=10 animals. Results were unequivocal in all samples. The effect was “all-or-nothing” as documented in FIGS. 3B, 4A, 4D, and 4E thereby yielding a binary outcome. All animals examined during the course of this project in our 9 laboratory were included in the report; no data to the contrary was encountered. To assess the type I error for the primary outcome, a sign test was performed, yielding a significance level of p<0.001. The primary endpoint of the study could thereby be answered definitively. The secondary objective of the study was to assess a functional outcome in the ACLT model, which was available only for a subset of animals, n=6. The model was newly established and can presently provide only a binary outcome based on the clinical assessment of gait asymmetry. Animal limping was compared before and after treatment by blinded observers labeling each subject as “improved” or “unchanged”. While all animals in the study were labeled correctly, i.e., all animals receiving RTX improved and all animals receiving vehicle control remained unchanged, the statistical power of this small set of observations with a binary outcome was only moderate, with p<0.02 based on the sign test. If considered in isolation, the behavioral results would provide only preliminary evidence of efficacy, however, additional support is provided by the agreement between the behavioral observations and the sP staining results.

Examples 1 through 6 demonstrate that RTX could exert its full effect via the PG/ED route as measured by a surrogate marker index, sP depletion, and alteration of chronic pain behavior. Furthermore, the Examples also provided evidence that PG-ED RTX improves clinically relevant functional outcomes related to pain. Positive results in the novel pig model of knee osteoarthritis pain characterized by asymmetry of weight bearing that mirrors the knee osteoarthritis symptoms of human patients provides evidence of pain diminution. In a blinded experiment, RTX led to significant reversal of gait asymmetry, thus demonstrating a functional outcome, with p<0.02.

### Example 7: Pain Reduction with Injection of 1.5 μg and 5 μg of RTX

A test was conducted on a second group of pigs to assess whether a lower dosage of drug would be effective. This test was conducted with the methodology set forth in Table 1 and described above and using epidural-periganglionic (PG-ED) injections. In this study, doses of 1.5 μg and 5 μg RTX were administered.

The data shows that treatment of pain can be achieved by a single, lower dose administration of RTX. A histology summary for sP results with a blinded observer found significance (P=0.002) with 6 of 6 control animals normal and 6 of 6 RTX dosed animals with low/none histology. PG-ED RTX improved clinically observed pain-related behavior in pigs with knee osteoarthritis (n=8) with a blinded observer (p=0.03). The data are provided in Table 1 below, where “Normal” indicates typical pain-related behavior.

TABLE 1
Observation of Histology and Pig Gait after Treatment

| Histology | | | | | |
|---|---|---|---|---|---|
| Summary | | | | | |
| (Blinded | Pain- | | | | |
| Observer | related | Pig | RTX/Vehicle | Needle | Sample |
| Call) | Behavior | ID | Dose | Type | IDs |
| No RTX | Normal | B379 | 5 μg/500 μL | 6″ 22G | SC-C8, |
| effect | | | (hand injected) | short bevel | SC-L5 |
| Ipsilateral | −−/− | B401 | 5 μg/500 μL | Epidural | SC-C8, |
| RTX effect | | | (hand injected) | catheter | SC-L4 |
| Normal | Normal | B402 | 1.5 μg/150 μL | 6″ 22G | SC-C8, |
| | | | (pump injected) | Quincke pt | SC-L5 |
| Normal | Normal | B404 | 5 μg/500 μL | 6″ 22G | SC-C8, |
| | | | (hand injected) | Quincke pt | SC-L5 |
| Diminished | −/− | B405 | 5 μg/500 μL | 6″ 22G | SC-C8, |
| sP | | | (pump injected) | Quincke pt | SC-L5 |
| Down | −/− | B406 | 5 μg/500 μL | 6″ 22G | SC-C8, |
| on both | | | (hand injected) | short bevel; | SC-L5 |
| side +/0 | | | | 3.5″ 22G | |
| | | | | Touhy | |
| Clearly | Normal | B408 | 5 μg/500 μL | 3.5″ 22G | SC-C8, |
| normal | | | (hand injected) | Touhy | SC-L5 |
| +/++ but | −/? | B409 | 5 μg/500 μL | 6″ 22G | SC-C8, |
| clear effect | | | (pump injected) | Quincke pt | SC-L5 |
| Effect | −−/− | B410 | 5 μg/500 μL | 6″ 22G | SC-C8, |
| | | | (hand injected) | Quincke pt | SC-L5 |
| Normal; | Normal | B411 | 5 μg/500 μL | 6″ 22G | SC-C8, |
| may need | | | (hand injected) | short bevel | SC-L5 |
| to recut it | | | | | |
| Normal | Normal | B414 | 1.5 μg/150 μL | 6″ 22G | SC-C8, |
| | | | (pump injected) | Quincke pt | SC-L5 |
| Obvious | −/−− | B415 | 5 μg/500 μL | 3.5″ 18G | SC-C8, |
| effect | | | (hand injected) | Touhy + | SC-L4 |
| | | | | 21G enidural | |
| | | | | catheter | |

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present disclosure is not entitled to antedate such publication.

While the present technology has been particularly shown and described with reference to example embodiments thereof, it will be understood by those skilled in the art that various changes in form and details may be made therein without departing from the scope of the invention encompassed by the appended claims.

1. A method of treating pain in a patient comprising:
identifying one or more locations within or proximate the patient's epidural space,
using imaging technology to direct an injection comprising resiniferatoxin to each of the one or more locations.

2. The method of claim 1, wherein the step of identifying the one or more locations comprises choosing specific dorsal root ganglia.

3. The method of claim 1, wherein the imaging technology is fluoroscopy based.

4. The method of claim 1, wherein the imagining technology is computer tomography based.

5. The method of claim 2, wherein the injection of resiniferatoxin to specific dorsal root ganglia induces cell apoptosis of TRPV1-expressing dorsal root ganglia.

6. The method of claim 6, wherein the cell apoptosis of TRPV1-expressing dorsal root ganglia reduces the expression of substance P.

7. The method of claim 1, wherein the injection comprises about 500 ng to about 50 μg of resiniferatoxin.

8. The method of claim 1, wherein the injection comprises about 1 μg to about 10 μg of resiniferatoxin.

9. The method of claim 1, wherein the injection comprises about 500 ng to about 3.0 μg of resiniferatoxin.

10. The method of claim 2, wherein the injection has a total volume of about 100 μl to about 1000 μl.

11. The method of claim 2, wherein the injection has a total volume of about 500 μl.

12. The method of claim 1, wherein the injection additionally comprises an analgesic.

13. The method of claim 12, wherein the analgesic is a local anesthetic.

14. The method of claim 1, wherein the injection additionally comprises 0.25% bupivacaine.

15. The method of claim 1, wherein the injection is delivered by infusion.

16. The method of claim 15, wherein the infusion is performed at a flow rate of about 10 μL/min to about 500 μl/min.

17. The method of claim 15, wherein the infusion is performed at a flow rate of about 100 μL/min.

18. The method of claim 1, wherein the one or more locations are a single location.

19. A method of treating pain in a patient comprising:
a) imaging the spinal target level of the patient;
b) inserting a spinal needle percutaneously into an epidural space proximate a lateral nerve ganglion of the patient;
c) verifying the position of the spinal needle by imaging or injection of contrast media; and
d) delivering resiniferatoxin to the epidural space.

20. The method of claim 19, wherein delivering resiniferatoxin comprises delivering resiniferatoxin at specific dorsal root ganglia.

21. The method of claim 19, wherein the imaging is by fluoroscopy.

22. The method of claim 19, wherein the imaging is by computer tomography.

23. The method of claim 20, wherein delivering resiniferatoxin at specific dorsal root ganglia comprises inducing cell apoptosis of TRPV1-expressing dorsal root ganglia.

24. The method of claim 23, wherein the cell apoptosis of TRPV1-expressing dorsal root ganglia reduces the expression of substance P.

25. The method of claim 19, wherein delivering resiniferatoxin comprises delivering about 500 ng to about 50 μg of resiniferatoxin.

26. The method of claim 19, wherein delivering resiniferatoxin comprises delivering about 1 μg to about 10 μg of resiniferatoxin.

27. The method of claim 19, wherein delivering resiniferatoxin comprises delivering about 500 ng to about 3.0 μg of resiniferatoxin.

28. The method of claim 19, wherein the resiniferatoxin is diluted in about 100 μl to about 1000 μl of diluent.

29. The method of claim 19, wherein the resiniferatoxin is diluted in about 500 μl of diluent.

30. The method of claim 28, wherein the diluent comprises an analgesic.

31. The method of claim 30, wherein the analgesic is 0.25% bupivacaine.

32. The method of claim 19, wherein delivering the resiniferatoxin comprises delivering the resiniferatoxin by infusion.

33. The method of claim 32, wherein the infusion is performed at a flow rate of about 10 μL/min to about 500 μl/min.

34. The method of claim 32, wherein the infusion is performed at a flow rate of about 100 μL/min.
